(19) United States
(12) Patent Application Publication
(10) Pub. No.: US 2012/0150299 A1
(43) Pub. Date: Jun. 14, 2012

(54) INTEGRATED MULTI-ZONAL CAGE/CORE IMPLANTS AS BONE GRAFT SUBSTITUTES AND APPARATUS AND METHOD FOR THEIR FABRICATION

(76) Inventors: Asli ERGUN, Hoboken, NJ (US); Dilhan Kalyon, Teaneck, NJ (US); Halil Gevgilili, Weehawken, NJ (US); Antonio Valdevit, Effort, PA (US); Arthur Ritter, Morristown, NJ (US)

(21) Appl. No.: 13/157,429

(22) Filed: Jun. 10, 2011

Related U.S. Application Data
(60) Provisional application No. 61/353,468, filed on Jun. 10, 2010.

(51) Int. Cl. A61F 2/44; B29C 47/06

(52) U.S. Cl. 623/17.11; 264/171.1; 425/4 C

(57) ABSTRACT

A surgical implant including a cage having a first porosity and a first modulus; and a core bounded by said cage, said core having a second porosity that is higher than said first porosity of said cage, and said core having a second modulus that is lower than said first modulus of said cage. The implant may be functionally graded in a transverse direction, a longitudinal direction, or a radial direction thereof. The implant is made by preparing a first formulation for the cage within a first extruder and a second formulation for the core within a second extruder, extruding the first formulation through a co-extrusion die while simultaneously extruding said second formulation through the co-extrusion die so as to form an extrudate that includes said cage component and said core component bounded by said cage component.

### CROSS-REFERENCE TO RELATED APPLICATIONS

This application is a §111(a) application relating to and claims the benefit of U.S. Provisional Patent Application Ser. No. 61/353,468, filed on Jun. 10, 2010, the disclosure of which is incorporated herein by reference in its entirety.

### FIELD OF THE INVENTION

The present invention relates to a bone graft substitute, and, more particularly, to an integrated multi-zone bioresorbable cage/core implant.

### BACKGROUND OF THE INVENTION

Currently, one of the most expensive health care problem in the US, with annual reported costs of $20 to $100 billion dollars, is the treatment of lower back pain (LBP). LBP is generally associated with the degeneration of the vertebral discs. Matrix composition, abnormal mechanical loading, genetic disposition, and reduced cell activity are some of the factors that can lead to such degenerative disc disease and could require surgery. Accordingly, there are over 200,000 spinal arthrodesis procedures carried out each year in the Unites States. Such spine fusion surgeries are responsible for the majority of bone-grafting procedures utilizing mainly autografts, and also allografts, xenografts, and synthetic materials as bone-grafting materials. One important surgical procedure involves anterior cervical discectomy with fusion for patients suffering back pain and/or neurological deficits. Such surgeries involve the removal of autologous bone (bone fragments harvested from the patient during the first part of the surgery) to be grafted into the spine to induce spine fusion after discectomy and removal of compressive structures. However, the nonunion rates associated with autologous bone removal and grafting is 5-35% and the donor site pain and morbidity are significant issues. Existing alternatives include allograft bone, bone marrow cells, porous calcium phosphates, demineralized bone matrix and bone growth factors.

Titanium cages have been used for some time but their use is diminishing due to the mismatch of mechanical properties with bone, causing corrosion and wear in the implant site, and the radiopacity of the metal to x-rays. Poly(ether ether ketone) PEEK is a polymer that has been used which is not biodegradable. For such implants, second surgery is sometimes required to remove the implant after the fusion is completed or when a repair or dislodgement is necessary. The long term effects of wear and degradation of PEEK polymer implants remains unknown at this time. While ceramics have also been used, the brittle nature has primarily confined them to arthroplasty use as bending and torsion can induce catastrophic failure. A non-crystalline polylactide copolymer, PLA, has also been used, however, PLA cages suffer from relatively poor mechanical properties and unpredictability associated with sudden hydrolysis and molecular weight cleavage effects that adversely affect their adaptation. The relatively high concentration of acidic degradation products of PLA leads to local inflammation and osteolysis, necessitating drainage and good vascularization of the tissues around the PLA implant. Furthermore, all of these implants display a single and isotropic modulus, which causes sharp changes in modulus between the implant and the host bone.

### SUMMARY OF THE INVENTION

In an embodiment, a surgical implant includes a cage having a first porosity and a first modulus, and a core bounded by the cage, the core having a second porosity that is higher than the first porosity of the cage, and the core having a second modulus that is lower than the first modulus of the cage. In an embodiment, the implant is adapted to be implanted into a host bone, such that the first modulus of the cage is selected to substantially match a modulus of a cortical bone portion of the host bone, and the second modulus of the core is selected to substantially match a modulus of a cancellous bone portion of the host bone. In another embodiment, the implant is adapted to be implanted into an intervertabral space. In other embodiments, the implant is functionally graded in a transverse direction, a longitudinal direction, or a radial direction thereof.

In an embodiment, the cage encapsulates the core. In another embodiment, the cage is concentrically formed around the core. In another embodiment, the cage includes a first layer and a second layer, with the core being sandwiched between the first and second layers of the cage. In an embodiment, the cage and the core are made from a bioabsorbable polymer, which may include poly(caprolactone). In another embodiment, the first porosity of the cage is about 74%, and the second porosity of the core is about 80%.

In an embodiment, a method of making a surgical implant comprising the steps of: preparing a first formulation for a cage component of the implant; preparing a second formulation for a core component of the implant; feeding the first formulation into a first extruder; feeding the second formulation into a second extruder; extruding the first formulation through a co-extrusion die while simultaneously extruding the second formulation through the co-extrusion die so as to form an extrudate that includes the cage component and the core component bounded by the cage component; conveying the extrudate from the co-extrusion die; and forming the extrudate to a desired size and shape. In an embodiment, the first formulation includes a bioabsorbable polymer and a first porogen, and the second formulation includes a bioabsorbable polymer and a second porogen. In another embodiment, each of the first and second porogens includes poly(ethylene glycol) and sodium chloride. In another embodiment, the first formulation comprises, by weight, about 20% poly(ethylene glycol), about 30% poly(caprolactone), and about 50% sodium chloride, and the second formulation comprises, by weight, about 36% poly(ethylene glycol), about 24% poly(caprolactone), and about 40% sodium chloride.

In an embodiment, an apparatus for making a surgical implant includes a first extruder for extruding the first formulation a second extruder for extruding the second formulation, and a co-extrusion die connected to the first extruder and the second extruder. In an embodiment, the co-extrusion includes a first channel in communication with the first extruder and a second channel in communication with the second extruder. In an embodiment, the first and second channels converge with one another so as to form a transition zone. In an embodiment, the first channel of the co-extrusion die is adapted to convey the first formulation through the transition zone, and the second channel of the co-extrusion die is adapted to convey the second formulation through the transition zone to form an implant extrudate.

### BRIEF DESCRIPTION OF THE DRAWINGS

For a more complete understanding of the present invention, reference is made to the following detailed description of exemplary embodiments considered in conjunction with the accompanying drawings, in which:

FIG. 1A is a cross-sectional view of a multi-zonal implant constructed in accordance with an embodiment of the present invention, showing an integrated cage and core configuration;

FIG. 1B is a cross-sectional view of a multi-zonal implant constructed in accordance with a second embodiment of the present invention, showing an integrated cage and core configuration;

FIG. 1C is a cross-sectional view of a multi-zonal implant constructed in accordance with a third embodiment of the present invention, the implant including an integrated cage and core configuration;

FIG. 1D is a cross-sectional view of a multi-zonal implant constructed in accordance with a forth embodiment of the present invention, the implant including an integrated cage and core configuration;

FIG. 1E is a cross-sectional view of a multi-zonal implant constructed in accordance with a fifth embodiment of the present invention, the implant including an integrated cage and core configuration;

FIG. 2A is a side elevational view of the multi-zonal implant the implant including in FIG. 1C;

FIG. 2B is a cross-sectional view of the multi-zonal implant taken along section lines 2B-2B of FIG. 2A, and looking in the direction of the arrows;

FIG. 3 is a schematic drawing and associated micrographs of the multi-zonal implant shown in FIG. 1C;

FIG. 4 is a perspective view of a transverse section of the multi-zonal implant shown in FIG. 1B;

FIG. 5 is micrograph of the multi-zonal implant shown in FIG. 1B;

FIG. 6 is a schematic view of an apparatus for the production of an extrudate from which the multi-zonal implants shown in FIG. 1A and is formed, the apparatus having a twin screw extruder and a ram extruder connected to a co-extrusion die;

FIG. 7 is a schematic drawing of the twin screw extruder connected to the co-extrusion die shown in FIG. 6, the twin screw extruder having a pair of counter rotating twin screws;

FIG. 8 is a schematic view of the counter rotating twin screws shown in FIG. 7;

FIG. 9 is a perspective view of the co-extrusion die shown in FIGS. 6 and 7, a portion of the co-extrusion die being depicted transparently to reveal flow channels formed therein;

FIG. 10 is a cross-sectional view of the co-extrusion die taken along the section lines 10-10 of FIG. 9, and looking in the direction of the arrows;

FIG. 11A depicts a portion of a human long bone with a tumor;

FIG. 11B depicts a segmental bone defect repair of the human long bone shown in FIG. 11A, using the implant shown in FIGS. 4, and 5;

FIG. 11C is a graph of the stress/strain relationships of the implant shown in FIGS. 4 and 5 and the human long bone shown in FIG. 11A;

FIG. 12 is a schematic view of an apparatus for the production an extrudate from which implants are formed, the implants having a cage with a gradation in the concentration of biphasic ceramic along their longitudinal axis and porosity gradation in their transverse direction, the apparatus having two twin screw extruders connected to a co-extrusion die;

FIG. 13 is a bar graph showing the concentration of biphasic ceramic at segments 1-5 along the longitudinal axis of the extrudate shown in FIG. 12;

FIG. 14 is a graph and associated micrographs of cell attachment and proliferation of human fetal osteoblast (Hfob) on a prototype implant;

FIG. 15 is a graph of Hfob cell differentiation on a prototype implant; and

FIG. 16 is a graph and an associated micrograph of mineralized matrix formation on a prototype implant.

### DETAILED DESCRIPTION OF THE EXEMPLARY EMBODIMENT

An integrated multi-zonal bioresorbable cage and core implant is provided for use as a bone graft substitute in a body. The implant has a stiff cage, with relatively low porosity, that encapsulates a core of higher porosity. The cage and the core are manufactured as an integrated part using co-extrusion from one or more biodegradable polymer(s). The polymer(s) incorporates various additives, mediators and drugs (i.e., for controlled release of same). The polymer(s) biodegrades in the body to be gradually substituted by bone tissue. The cage/core implant can serve also as a scaffold for conversion into a tissue construct prior to implantation in the body, upon cell seeding/proliferation/differentiation in a bioreactor preferably using the patient's own harvested cells. The cage/core implant can also be functionally graded along its longitudinal axis (e.g., by altering its composition and/or porosity along the longitudinal axis).

The implant is bioabsorbable and has graded physical characteristics for matching the implant modulus to that of the host bone, thereby reducing the sharp change in modulus between the implant and the host bone. In the case of an implant-bone interface, the sharp change in modulus as in the case of metal implants can lead to increased stress induced microfracture. While a single value of the modulus can be integrated into an implant comprised of a polymeric material, in orthopedic applications, the host bone does not consist of a single isotropic material. The adjacent bone will consist of two bone types: cancellous and cortical. The implant provides at least two moduli that enhance the matching of physical characteristics of the host bone, such as a lower modulus for reduction of stress shielding where cancellous bone is contacted and sustained load bearing via an increased modulus where cortical bone is adjacent to the implant. More particularly, the cortical and cancellous bone types display unique material and mechanical properties. Thus, the implant provides modulus matching capability as well as account for the varying isotropic properties such as porosity and load bearing.

The cage/core implant can be used as bone graft substitute. Characteristics of the integrated cage/core implant are easily adapted to numerous applications (i.e., the stiffness, porosity, mechanical properties, and biodegradation rates can be tailored depending on the location of the implantation). Furthermore, the integrated cage/core implant can be used as a tissue engineering scaffold. This requires harvesting appropriate cells either from the patient or from other sources, to be proliferated and seeded into the scaffold to generate a tissue construct that is then implanted into the patient to facilitate faster regeneration of the bone tissue at the implant site.

The implant allows modulus matching for cortical and cancellous bone types via the co-extrusion method. The internal polymer can be tailored to display a reduced modulus that is comparable to that found in cancellous bone. Such a property can be achieved via methods that alter polymer orientation, porosity and addition of secondary materials such as bone morphogenic proteins (BMPs). The inner porosity can be of importance with respect to the transportation of nutrients and signaling for any cells that may be deposited during healing or applied prior to implantation. The outer layer of the implant can be formulated so as to display a substantially increased modulus as compared to the inner region of the implant, thereby allowing for load bearing and tailoring the modulus closer to that of cortical bone. As with the inner polymer, the appropriate porosity of the outer layer can facilitate fluid flow, and, with healing, also aid in the process of angiogenesis to stimulate vascularity within the healing tissue. The implant can be fabricated so as to achieve modulus matching to cortical and cancellous bone types, and porosity can be adjusted to achieve radial modulus variations as well as provide nutrient flow channels.

Radial (or in the transverse direction in the case of rectangular scaffolds) porosity, pore size and/or bioactive agent/s (such as but not limited to bioactive ceramics, i.e. hydroxyapatite (HA) and tricalcium phosphate (TCP), growth factors, drugs) gradations are achieved using a co-extrusion die. Two or more continuous twin screw or ram (or a combination) extruders feed two or more streams with differing formulations into a co-extrusion die where they merge together forming a single, continuous, multi layered structure which is profiled into a final desired shape. By changing the formulations comprising the different layers, the porosity, pore size and/or bioactive agent/s gradations in the radial and/or transverse directions is achieved. The final product can be shaped into a cylinder with several layers in the radial direction, or into a rectangular strand with multiple layers or with a core structure. To generate different shapes and gradations, the flow channels of the co extrusion die and number of streams feeding to die can be altered.

The aforesaid description is provided in general terms. The implant, the method and apparatus for making the implant, and applications for using the implant are now described in detail hereinbelow.

Integrated Multi-Zonal Cage/Core Implant with Radial Gradation

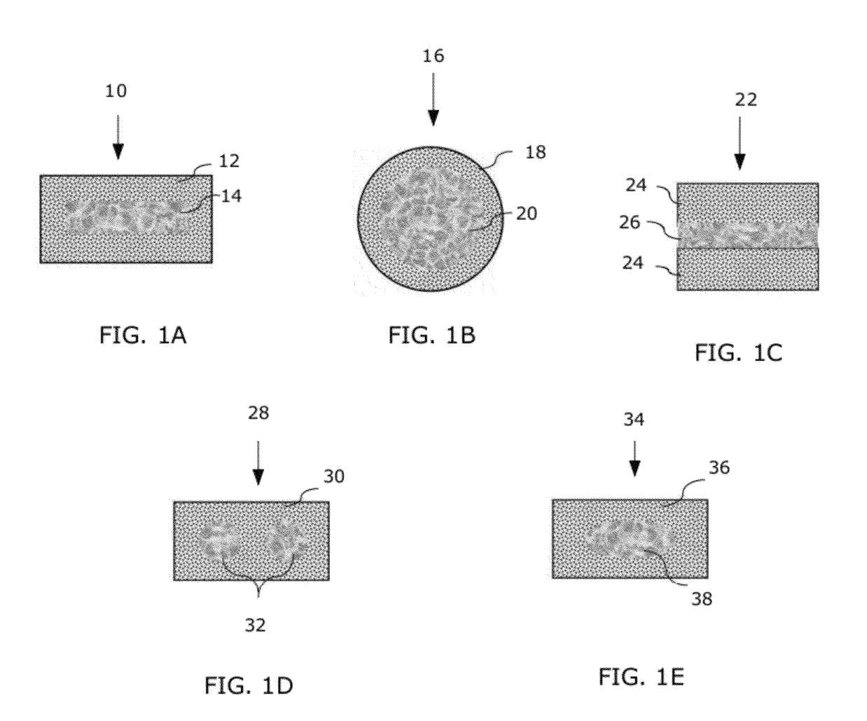

FIGS. 1A-1E illustrates embodiments of integrated multi-zonal cage/core configurations that can be achieved with minor tooling modifications to the co-extrusion die (which will be described in further detail below). For instance, FIG. 1A shows an Implant 10 that has a cage 12 that encapsulates a core 14. FIG. 1B shows an implant 16 which has a cage 18 that is concentrically formed around a core 20. FIG. 1C illustrates an implant 22 that has a core 26 that is sandwiched between two layers of cage 24. FIG. 1D depicts an implant 28 that has a cage 30 positioned around two tubular-shaped cores 32. FIG. 1E shows an implant 34 that has a cage 36 that encapsulates an oval shaped core 38.

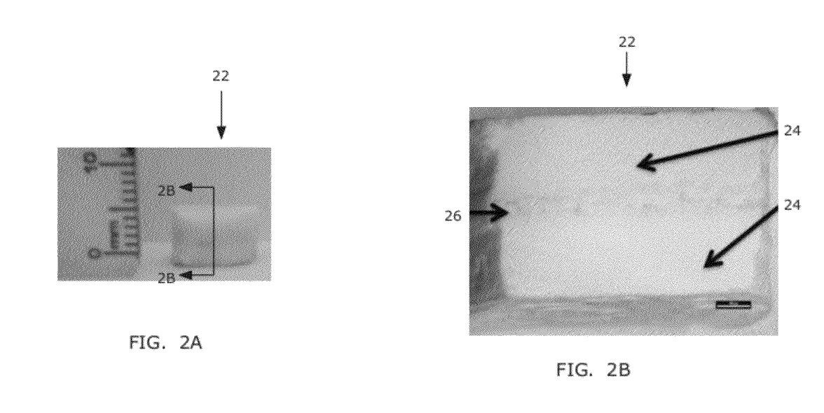

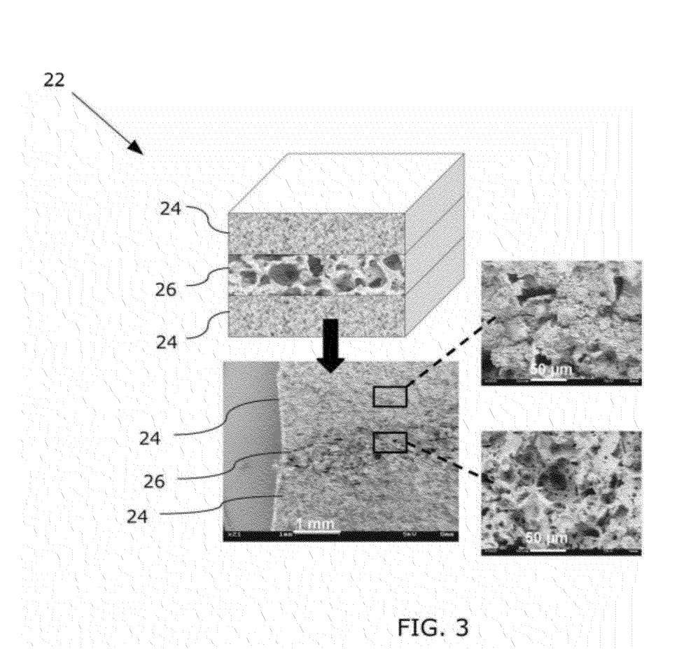

By incorporating different types, particle sizes and concentrations of porogens and/or bioactive agent/s into the cages and cores described above, gradation of physical properties in the radial or transverse direction can be tailored as desired depending on the application. For instance, the implants 10 and 22 are applicable to spinal fusion. FIGS. 2A and 2B and FIG. 3 show additional details of the implant 22, the increased porosity of the core 26 being evident in comparison to the porosity of the cage 24.

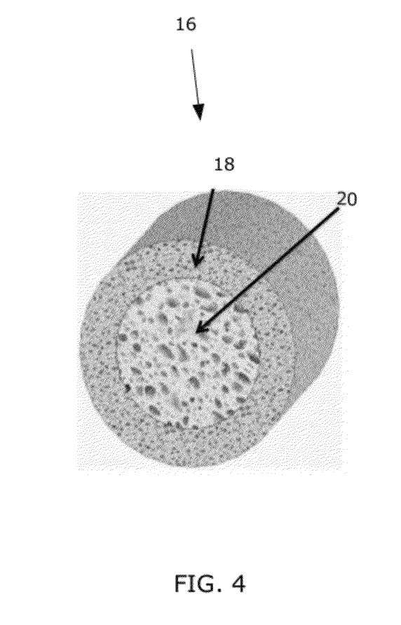

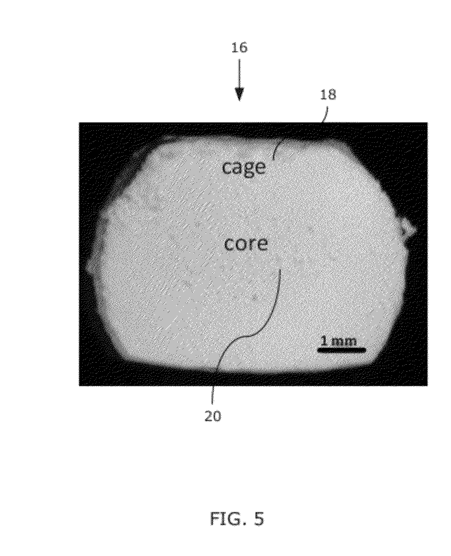

In another application, the implant 16 is applicable to the repair of segmental bone defects. FIGS. 4A and 2B and FIG. 5 show additional details of the implant 16, the increased porosity of the core 20 being evident in comparison to the porosity of the cage 18.

Apparatus for Fabricating the Implant

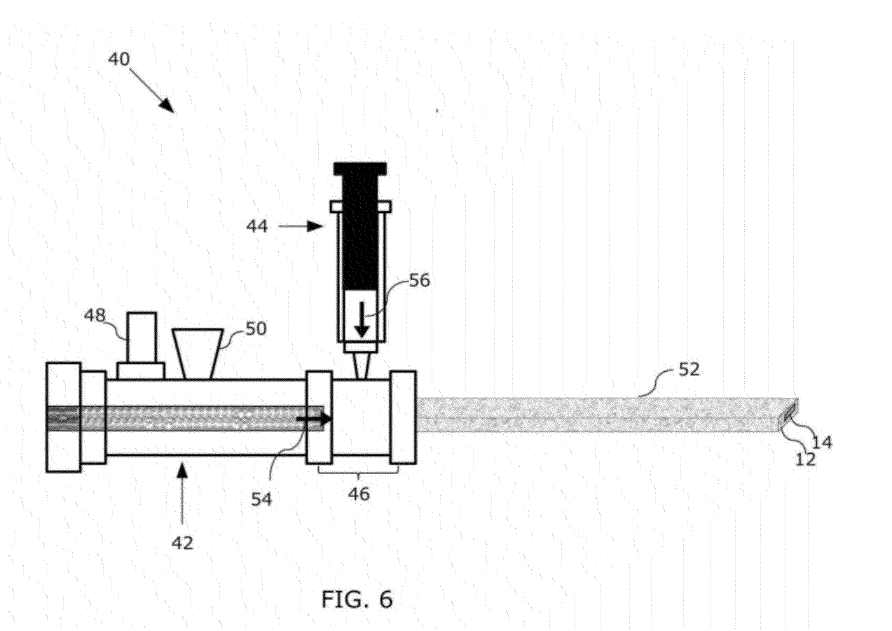

FIG. 6 illustrates a co-extrusion apparatus 40 which has a twin screw extruder 42, a ram extruder 44 and a co-extrusion die 46. The twin screw extruder 42 has a first feeding port 48 and a second feeding port 50. In an embodiment, the co-extrusion apparatus 40 is used to fabricate a multi-layered extrudate 52. The multi-layer extrudate 52 is an elongated formation from which, in this embodiment, the implants 10 are formed (e.g., shaped, cut away, etc.). A formulation 54 for the formation of the cage 12 is extruded from the twin screw extruder 42, and a formulation 56 for the formation of the core 14 is extruded from the ram extruder 44, and both are simultaneously fed to the co-extrusion die 46 from which emerges the extrudate 52. The formulations 54, 56 are each composed of multiple ingredients that are mixed together depending on the requirements of cage 12 and the core 14, respectively, of the implant 10. Adequate pore size and porosity distributions for the cage 12 and the core 14 are achieved either by using a solid porogen, like salt or a polymer that is later dissolved, or through the incorporation of gases (including gas(es) under their supercritical condition(s)). The multi-layered extrudate 52 utilizes a water-soluble double porogen system, i.e., poly (ethylene glycol) (PEG, molecular weight: 35000 g/mol) and sodium chloride (NaCl) particles, particle size ranging about 45 μm to 180 μm incorporated into poly(caprolactone), PCL, at different concentrations for the formulation 54 and the formulation 56 to constitute the cage 12 and the core 14, respectively, of the extrudate 52. More particularity, the PEG/PCL/salt concentrations used in the formulation 54 and the formulation 56, are approximately 20/30/50% and 36/24/40%, by weight, respectively, to tailor the porosity and interconnectivity of the different layers of the extrudate 52 to achieve different pore size and porosity distributions for the cage 12 and the core 14 layers. The porosity of the extrudate 52 components are about 74% for the cage 12 and about 80% for the core 14. The extrudate 52 has an elastic modulus of about 0.25 GPa under compression.

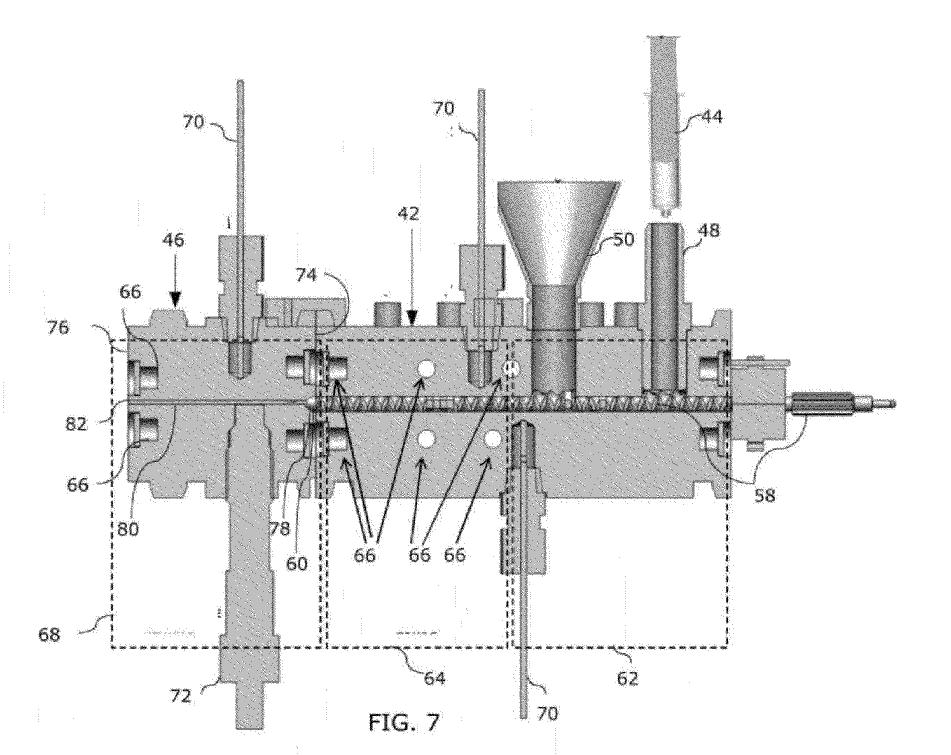

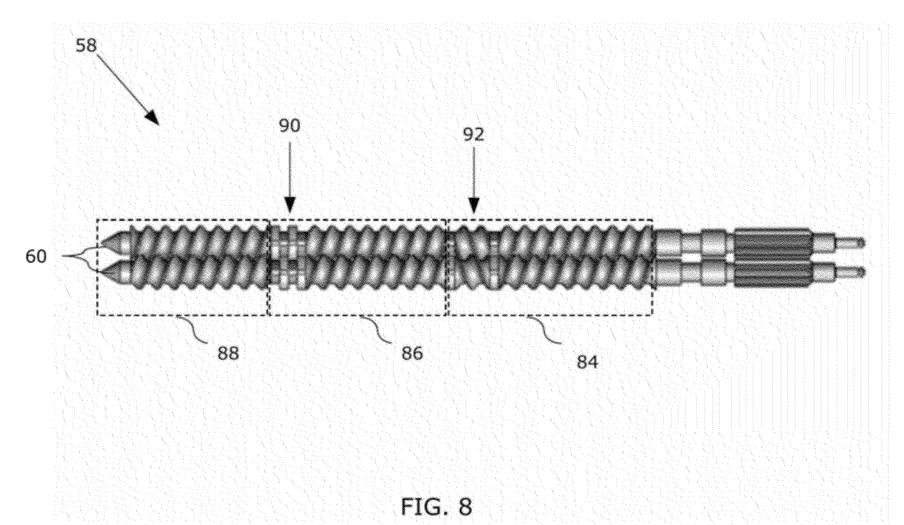

FIG. 7 depicts the twin screw extruder 42 connected to the co-extrusion die 46. The twin screw extruder 42 has a pair of counter-rotating screws 58. The counter-rotating screws 58 have pointed ends 60 (see FIG. 8). The first feeding port 48 of the twin screw extruder 42 is connected to the counter rotating screws 58 for accepting a polymer melt feed mixture of PCL and PEG, and the second feeding port 50, is connected to the screws 58, for accepting salt (i.e., to form the formulation 54). The twin screw extruder 42 has a first barrel heating zone 62, a second barrel heating zone 64, through constant temperature fluid circulation channels 66 for circulating hot oil (not shown) throughout the twin screw extruder 42 to maintain it at a predetermined temperature. Likewise, the co extrusion die 46 has oil circulation channels 66 and a heating zone 68 for circulating hot oil to maintain co-extrusion die 46 at a predetermined temperature. Temperature probes 70 and a pressure transducer 72 are positioned in the extruder 42.

The co-extrusion die 46 has an inlet end 74 that is attached to the twin screw extruder 42, and a discharge end 76 that is positioned opposite the inlet end 74. A cone shaped convergence zone 78 is positioned proximate the end 74. The co-extrusion die 46 has a first channel 80 that extends longitudinally from the convergence zone 78 to an extrudate discharge opening 82 located at the discharge end 76, for purposes that are described hereinbelow.

Referring to FIG. 8, the counter rotating screws 58 has a first mixing zone 84, a second mixing zone 86, a third mixing zone 88, with four pairs of neutral kneading disks 90 in the second mixing zone 86 and a combination of ninety degree neutral kneading disks and fully flighted reversing elements 92 in the first mixing zone 84. The counter rotating screws 58 are described in greater detail hereinbelow.

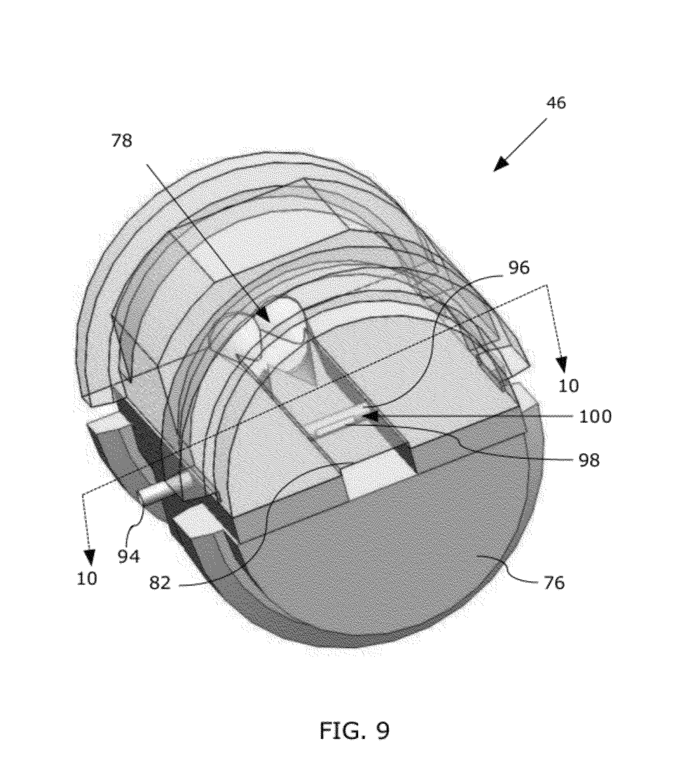

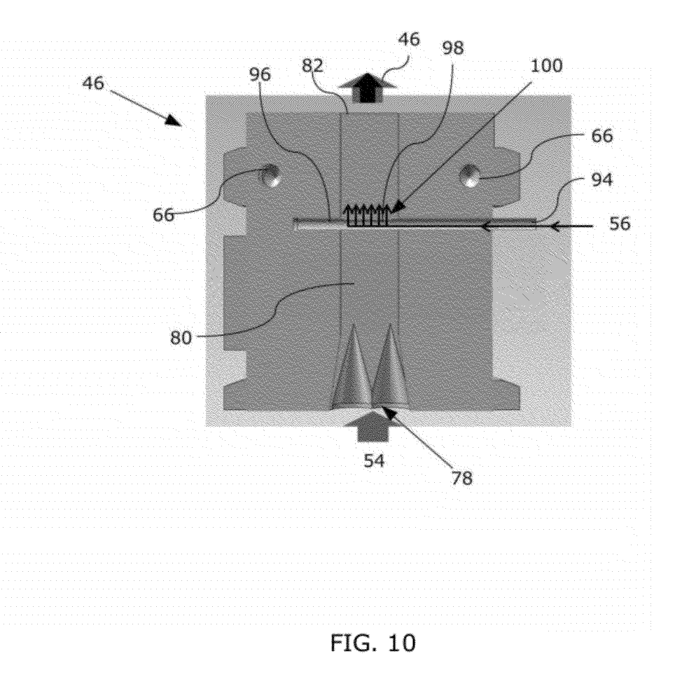

Referring to FIGS. 9-10, the convergence zone 78 of the co-extrusion die 46 is sized, shaped, and positioned around the pointed ends 60 (see FIG. 8) of the counter rotating screws 58 so such that the formulation 54 (see FIG. 6) flows uniformly through the convergence zone 78 into the first channel 80. The co-extrusion die 46 has a side feeding port 94 which is connected to the ram extruder 44 (or a second twin screw extruder 42). A tubular-shaped second channel 96, which has a rectangular discharge slit 98, extends from the side feeding port 94 and intersects the first channel 80 at a right angle, forming an integration zone 100. The formulation 56 (see FIG. 6) flows through the discharge slit 98 (see FIG. 10) of the second cannel 96 into the transition zone 100, thereby adhering to the formulation 54 and forming the integrated multi-zonal extrudate 52, which is discharged out of the extrudate discharge opening 82.

Since the formation of the extrudate 52 is under pressure, the cage 12 and core 14 adhere to each other. There is no longitudinal length limitation for the extrudate 52, so as not to be limited for use as a bone graft substitute (i.e., a spinal fusion implant). The length can be tailored depending on the application at no additional cost for tool design or additional implant 10 fabrication steps. Channels (not shown) can be opened up at desired locations (with location changes easily accomplished) by the inclusion of pins into the co-extrusion die 46. Upon implantation of the implant 10, the channels may allow blood flow or effluent removal during biodegradation of the implant 10.

Method for Fabricating the Implant

Referring to FIG. 6, the twin screw extruder 42 (e.g., a 7.5 mm L/D=15 co-rotating fully-intermeshing twin screw extruder Model: MPR ME7.5, Material Processing & Research, Inc. of Hackensack, N.J.) with complex screws and the ram extruder 44 in conjunction with a co-extrusion die 46 are utilized to fabricate implant 10. More particularly, the core formulation 56 comprising of 24 wt. % PCL/36 wt. % PEG/40 wt. % salt with particle sizes in the range of about 45 microns to 180 microns is first blended, for example, in Haake Rheocord Torque Rheometer and Haake Rheomix 3000E mixing chamber with counter rotating roller blades (not shown) at 90° C. and 32 rpm for 30 min to form as a preblend. The preblend is fed into the co-extrusion die 46 from the side feeding port 94 using the ram extruder 44 (e.g., Model PHD4400 BS4 programmable syringe pump, Harvard Apparatus, Holliston, Mass.) at a flow rate of 22.5 g/h. The temperature of the feed stream is maintained at 73° C. by oil circulating through the ram extruder 44.

The cage 14 is compounded in the twin screw extruder 42 and fed to the co-extrusion die 46. A polymer blend comprising of 60 wt. % PCU40 wt. % PEG is first blended in a Haake Rheocord Torque Rheometer and Haake Rheomix 3000E mixing chamber with counter rotating roller blades at 90° C. and 32 rpm for 15 min. The preblended polymer is first melted at 73° C. in a second ram extruder 44 by oil circulation and fed into the twin screw extruder 42 via the first feeding port 48 using a second ram extruder (e.g., Model: PHD4400 BS4 programmable ram extruder, Harvard Apparatus, Holliston, Mass.) at a flow rate of 60 g/h. Salt with particle sizes 45-90 microns is fed from the second feeding port 50 of the twin screw extruder 42 with a Brabender (Model: Brabender mini twin screw feeder DDSR12) lost-in-weight mini twin-screw solid feeder (not shown) with internal agitation at flow rate of 60 g/h. The polymer blend and the salt are mixed within the confines of the first two mixing zones 84 and 86 of the twin screw extruder 42. The mixing is achieved by using a combination of the kneading disks 90 and fully flighted reversing elements 92. The first mixing zone 84 has a the pair of 90°, neutral kneading blocks and fully flighted reversing elements 92. The second mixing zone, where salt is dispersed in the PCL-PEG polymer blend, has the 4 pairs of ninety degree neutral kneading disks 90. The configuration of the counter rotating screws 58 can be altered by changing the number and stagger angle of the kneading discs 90, 92 or utilization of a wide variety of screw elements to achieve the necessary degree of dispersive and distributive mixing as required. The third zone 88, located at the discharge of the extruder barrel, is used for deaeration and pressurization of the suspension to overcome the pressure drop through the co-extrusion die 46. The twin screw extruder 42, the ram extruder 44 and the co-extrusion die 46 are all maintained at 71-74° C. with constant temperature oil flow through the oil circulation channels 66. Temperatures can be altered for different polymers depending on their transition, i.e. softening/melting characteristics. The co-extrusion die heating zone 68 and the first and second barrel heating zones 62, 64 of the twin screw extruder 42 accommodate desired temperature profiles at different zones of the twin screw extruder 42 and the co-extrusion die 46. At 140 rpm screw speed, about 12-13% of the torque in the twin screw extruder 42 is exerted and about 5-6 psi pressure drop occurs through the co-extrusion die 46.

Use and Application of the Implant

The length of the implants 10 can be changed by cutting the multi-layered extrudate 52 that is continuously extruded to the desired length. The implants 10, upon the removal of the porogens using suitable means, may be sterilized and packaged/sealed for use as bone graft substitutes.

Regardless of which application is utilized, the basis of implantation is comparable. The insertion process involves removal of degenerative or diseased tissue to create a region where the implant may be placed in a predominantly compressive loading condition. The implant is then customized by the surgeon prior to implantation. Customization may involve cell seeding, BMP loading and sizing. A tensile distractive force is applied across the region where the implant is to be inserted followed by insertion of the implant resulting in a compressive force upon the implant resulting in stability and resistance to migration.

### Spinal Fusion Application:

The vertebral body supports approximately 80% of the load transmitted across a spinal segment. The intervertebral disc, which resides between the vertebral bodies and aids in this load support process, can begin degeneration after age 20. When degeneration becomes severe through trauma, lifestyle or natural aging, the disc may lend itself to dehydration (loss in intervertebral height), bulging or herniation (nerve impingement). Regardless of the condition, removal of the disc is performed when spinal fusion is indicated. The stabilizing and restoration of intervertebral height usually results in pain reduction. Spinal fusion involves placement of screws through the pedicles and creating a construct by interconnecting the screws with rods. While such a procedure addresses the posterior aspect and can reduce instability, it does not restore intervertebral disc height, and can lead to increased loading upon the facet joints as the spine can be placed in increased lordosis. The above complication is remedied by an anterior procedure involving removal of the intervertebral disc and insertion of the implant 10 between the intervertebral bodies prior to the posterior procedure. This allows the surgeon to compress the spine upon the implant 10 resulting in a more natural lordotic condition and hence a more mechanically balanced condition that is closer to the physiological environment.

The implantation of the implant 10 begins with a complete removal of the intervertebral disc (discectomy) resulting in exposure of the vertebral body surfaces that were in contact with the disc. The surgeon then inserts a distractor tool and opens the intervertebral disc space to a desired level. The surgeon then cuts the implant 10 to the desired size (with an increase in height if a press fit is considered). The modulus mating capability of the implant 10 allows for it to reside across the vertebral body endplate without the risk of subsidence that may occur with a stiff implant which makes contact with the softer inner region. The softer inner core 14 can deform under over loading, and the risk of subsidence is reduced as the high modulus outer core 12 sustains the load bearing required for immediate stability and height restoration. The implant 10 can be rendered radiolucent. Thus, the progress of the implant 10 in vivo can be readily examined because it is radiolucent. The implant 10 can be integrated with radio marker dots (not shown) so a surgeon can see where the implant 10 meets the vertebral body endplate.

### Segmental Bone Defect Application:

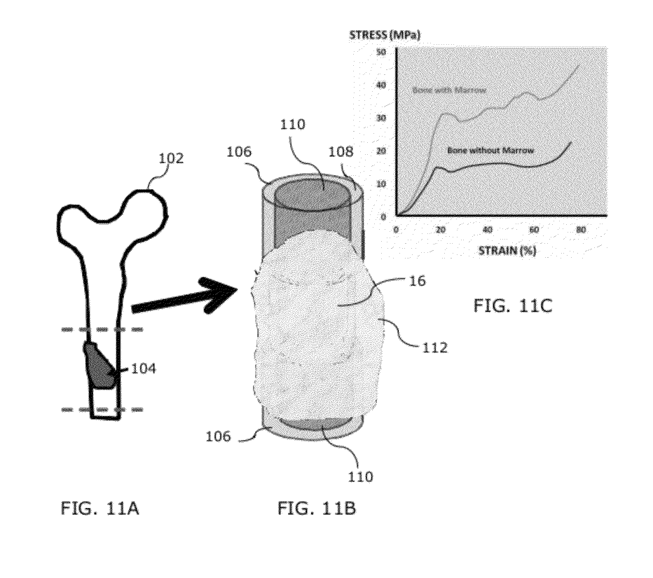

Referring to FIG. 11A, the implant 16 (see FIGS. 1, 4A, 4B, and 5) is applicable for bone repair of the diaphysis of a long bone 102 having, for example, a tumor 104. More particularly, FIG. 11B illustrates the cortical bone component 106, cancellous bone component 108, and the bone marrow component 110 of the host long bone 102, and the formation of callus and calcification 112 surrounding the implant 16. The implant 16 provides mechanical integrity for load bearing, yet allows for the marrow component 110 within the long bone 102 to facilitate healing. Porosity variations also allow for the marrow component 110 to function following implant 16 insertion. The inner core 14 with its high porosity allows nutrients and a cascade of cells to initiate the repair process, permeate the site and begin the repair and osteointegration. The stability of the outer core 12 which is matched to the modulus of the cortical bone component 106 minimizes the risk of stress shielding and promotes the initiation of callus and calcification 112. FIG. 11C indicates the modulus matching facilities of the implant 16.

Integrated Multi-Zonal Cage/Core Implant with Longitudinal Gradation

An implant can also be rendered functionally graded in its longitudinal direction to provide gradations in formulations to enable the development of three dimensional concentration gradients in medicinal drugs, growth factors, distributions of types and concentrations of fillers (including nanohydroxyapatite and tricalcium phosphate), and controlled variations in the biodegradation rates of the polymeric matrices and porosity.

Further longitudinal gradation of the co-extruded multilayer graded structure can also be achieved by altering the composition of the streams feeding to the co-extrusion die 46 as a function of time. This can be achieved by changing the flow rate of the individual streams feeding to the extruder/s, in the manner described hereinbelow.

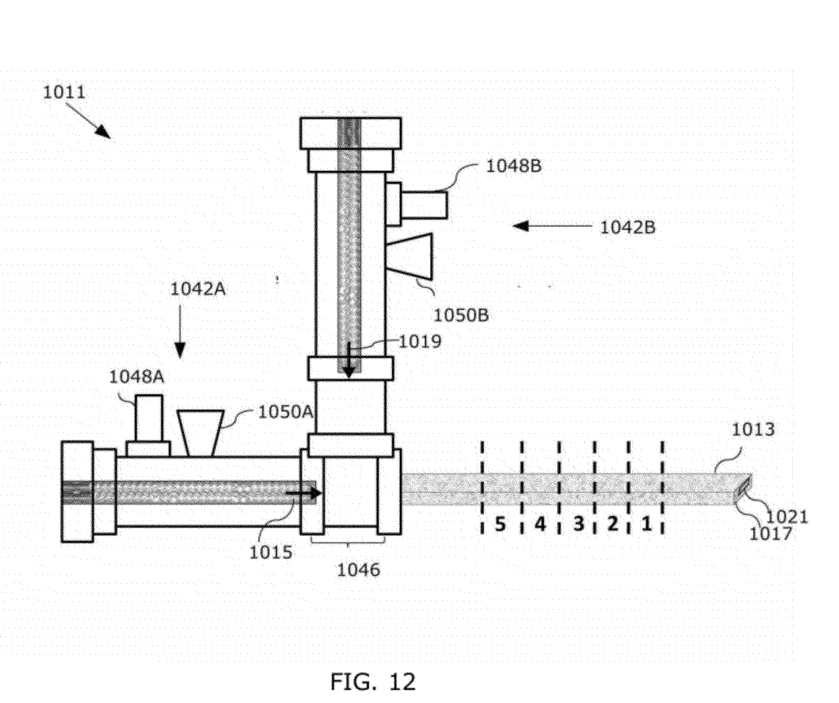

FIG. 12 depicts a second embodiment of the present invention. Elements illustrated in FIG. 12, which correspond, either identically or substantially, to the elements described above with respect to the embodiment shown in FIG. 6 have been designated by corresponding reference numerals increased by one thousand. Unless otherwise stated, the embodiment of FIG. 12 is constructed and assembled in the same basic manner as the embodiment of FIG. 6.

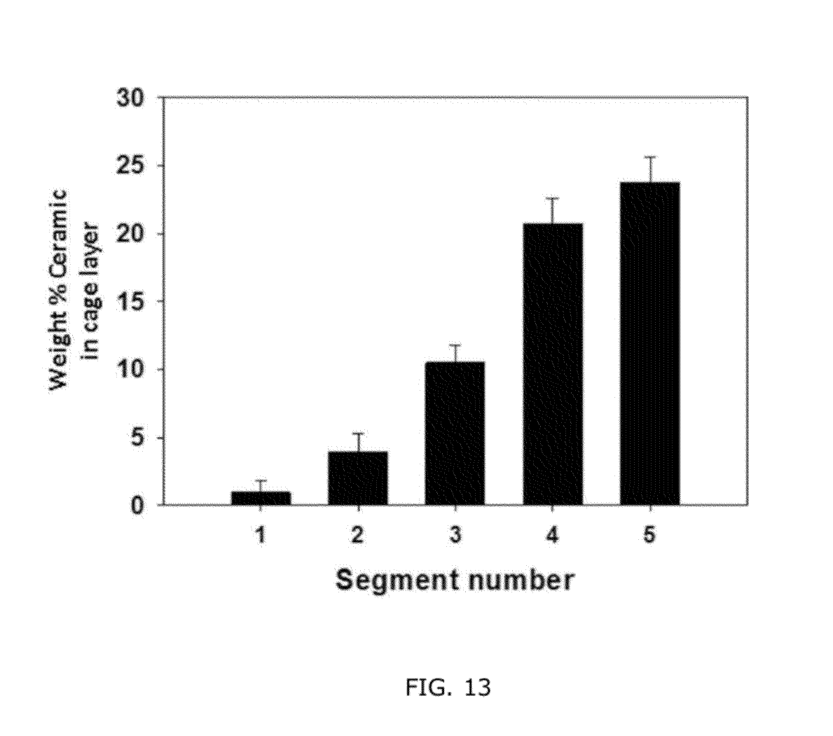

FIG. 12 illustrates a co-extrusion apparatus 1011 which has a pair of twin screw extruders 1042A, and 1042B, and a co-extrusion die 1046. The twin screw extruder 1042A has a first feeding port 1048A and a second feeding port 1050A. The twin screw extruder 1042B has a first feeding port 1048B and a second feeding port 1050B. The co-extrusion apparatus 1011 is used to fabricate a multi-layered extrudate 1013. The multi-layer extrudate 1013 is an elongated formation from which implants (not shown) are formed (e.g., shaped, cut away, etc.). A formulation 1015 for the formation of the cage 1017 of the extrudate 1013 is extruded from the twin screw extruder 1042A, and a formulation 1019 for the formation of the core 1021 of the extrudate 1013 is extruded from the twin screw extruder 1042B, and both are simultaneously fed to the co-extrusion die 1046 from which emerges the extrudate 1013. FIG. 12 shows five dashed vertical lines (1-5) depicting sections of the extrudate 1013 that are selected to illustrate the longitudinal gradation % weight of ceramic in the cage 1017 of the extrudate 1013, as shown on the bar chart shown in FIG. 13.

Each of the twin screw extruders 1042A, 1042B, which can be, for example, a 7.5 mm L/D=15 co-rotating fully-intermeshing MPR ME7.5 twin screw extruder (Material Processing & Research, Inc. of Hackensack, N.J.) with complex screws, and in conjunction with a co-extrusion die 1046 are utilized to fabricate an extrudate 1013 with a cage 1017 that is graded in the longitudinal direction. The outer cage layer formulation 1015 is processed in the twin screw extruder 1042A, and inner core layer formulation 1019 is processed by the twin screw extruder 1042B. The formulation 1015 and the formulation 1019 are fed to the co-extrusion die 1046. Thus, the co-extrusion die 1046 allows the feeding of two separate melt streams under pressure to form the extrudate 1013 from which implants (not shown) are formed. The formulation 1015 of the cage layer 1017 is altered systematically by changing the operating conditions of the twin screw extruder 1042A as a function of time, which provides the longitudinal gradation to the cage layer 1017. Hence, the porosity and pore size gradation of the outcoming implant changes both in transverse and longitudinal directions. The formulation 1019 of the core layer 1021 and the extrusion conditions are maintained the same as those of the core layer 14 of the implant 10 and hence the core 1021 is not longitudinally graded. However, changing the operating conditions of the twin screw extruder 1042B can also enable longitudinal gradation in the core layer 1021.

Exemplary Processing Conditions of the Longitudinally Graded Cage Layer 1017 (Twin Screw Extruder 1042A):

The outer cage layer formulation 1015 comprising of 30 wt. % PCL/20 wt. % PEG/50 wt. % salt with particle sizes 45-90 μm is first blended in a Haake Rheocord Torque Rheometer and Haake Rheomix 3000E mixing chamber (not shown) with counter-rotating roller blades at 90° C. and 32 rpm for 30 min to form a preblend. The preblended formulation is first melted at 73° C. in a ram extruder (not shown) by oil circulation and fed into the twin screw extruder 1042A via the first feeding port 1048A using a ram extruder (e.g., Model PHD4400 BS4 programmable syringe pump, Harvard Apparatus, Holliston, Mass.) at a flow rate of 110 g/h. The process is brought to steady-state operation (e.g., over a period of a few minutes) and biphasic calcium phosphate powder (containing 20 wt. % HA and 80 wt. % β-TCP) feeding is started at 10 g/h via the second feeding port 1050A on the twin screw extruder 1042A with, for example, a Brabender (Model: DDSR12) lost-in-weight mini twin-screw solid feeder (not shown) to enable biphasic calcium phosphate gradation in the longitudinal direction of the cage layer 1017. The feeding is started at time zero and stopped after 5 minutes to provide systematic gradation with increasing and decreasing biphasic ceramic phosphate concentration in longitudinal direction. Segments of the co-extruded extrudate 1013 are collected systematically and biphasic calcium phosphate gradation from 0% to 24 wt. % in axial direction in the cage layer is achieved upon leaching out the porogens (see FIG. 13). The temperature is kept at about 71-74° C. in both twin screw extruders 1042A and 14042B, and the co-extrusion die 1046 during the processing.

Exemplary Processing Conditions of the Inner Core Layer 1021 (Twin Screw Extruder 1042B):

The inner core layer 1021 is compounded in the twin screw extruder 1042B at 100 rpm and fed to the co-extrusion die 1046 at 20 g/h. A polymer blend comprising of 40 wt. % PCL/60 wt. % PEG which is blended in a Haake Rheocord Torque Rheometer and Haake Rheomix 3000E mixing chamber (not shown) is first melted at 73° C. in a ram extruder (not shown) by oil circulation and fed into the twin screw extruder 1042B via the first feeding port 1048B using a ram extruder (e.g., Model: PHD4400 BS4 programmable syringe pump, Harvard Apparatus, Holliston, Mass.—not shown) at a flow rate of 12 g/h (FIG. 3). Salt with particle sizes in the range of about 45 μm to 180 μm is fed from the second solids feeding port 1050B of the twin screw extruder 1042B with a second Brabender (Model: Brabender mini twin screw feeder DDSR12) lost-in-weight mini twin-screw solid feeder (not shown) at a flow rate of 8 g/h. The operating conditions of twin screw extruder 1042B are not altered during the processing.

The present invention provides functional gradations in chemical composition and properties in tissue engineering constructs in a reproducible and controllable manner which are crucial in attempts to mimic the natural gradations in various endogenous tissues for which the tissue engineering constructs are aimed. Conventional scaffolding methods do not generate gradations in composition and shape in a three dimensional manner.

It should be noted that the present invention can have numerous modifications and variations. For instance, various ingredients for various embodiments of the implant 10 may be kept in different feeders (not shown), all connected to a single or twin screw extruder or other delivery and pressurization apparati. Some of the ingredients may be liquid (for example the solvents) and some of the ingredients may be solid (for example, biodegradable polymers like poly(lactic acid) particles). The solid ingredients are typically kept within the hoppers (not shown), if necessary, under a blanket of an inert gas. The liquid ingredients are kept in reservoirs connected to various types of liquid pumps.

In an embodiment, solid feeders (not shown) are typically loss-in-weight or volumetric type and the liquid ingredients are fed using various types of pumps including gear pumps, centrifugal pumps, piston pumps etc. The feeding rates of various ingredients can be altered systematically with time.

The twin screw extruder 42 is used to bring all of the ingredients together to generate the material of construction of the scaffold to be shaped. The screw extruder 42 can change the phase of the various ingredients (by melting or, dissolution), for example by changing the temperature of the fluid using conduction through the barrel walls and viscous energy dissipation upon the conversion of the mechanical energy supplied through the rotating shaft/shafts or upon dissolution using appropriate solvent or solvents (not shown).

In an embodiment, the screw extruder 42 can be of the single or twin screw extruder type. For the twin screw extrusion mode, the screw extruder can have two screws which intermesh (fully-intermeshing upon the flank of one screw wiping the root of the other screw) or not intermesh (tangential) or any degree of intermesh in between these two extremes. The screws can rotate in the same direction or they can rotate in opposite directions (i.e., co-rotation versus the counter rotation). The screw elements are generally modular (consist of fully-flighted screws which are right or left handed, dispersion elements including kneading discs configured at differing stagger angles and stagger directions) generally with different screw combinations giving rise to very different processing capabilities (each different screw/barrel configuration provides a different continuous processor).

In an embodiment, the screw extruder 42 can be comprised of multiple mixing zones which, in turn, can be comprised of partially full and completely full sections with the degree of fill alterable on the basis of geometry, material properties and operating conditions. In an embodiment, the screw extruder 42 can have a sealed section at which vacuum can be drawn to remove the air content or to remove some of the excess solvents. The screw extruder 42 may have a fully-flighted right handed section (not shown) at the distal end of the screw to generate the pressure necessary for the material of construction of the scaffold to be extruded through the co-extrusion die 46.

In another embodiment the different flow streams that constitute the different layers of the multi-layered extrudate 52 can be mixed elsewhere (using batch or continuous mixers) and then fed using gear pumps or other melt delivery/pressurization apparati. Combinations of extruders and other melt delivery and pressurization apparati are also possible.

The co-extrusion die 46 allows multiple flow streams to merge under pressure. The design and the fabrication of the co-extrusion die 46, to generate the desired multilayer shape, requires careful analysis including numerical simulation in conjunction with the detailed characterization of the flow streams including their rheological and thermal behavior.

During operation, the screw extruder 42 typically melts, mixes, devolatilizes, pressurizes, and shapes the material of construction of the scaffold (i.e., the implant 10). Porosity, i.e. typically about 50 to 80%, in the multi-layered extrudate 52 emerging from the co-extrusion die 46 can be obtained by the use of porogens with differing concentrations (about 50-80% by volume if solid and dissolvable porogens are used) to generate interconnected porosity with pore sizes typically in a range of about 5 to 300 microns. If solid dissolvable porogens are utilized, they need to be removed in a secondary operation. The use of a chemical/physical blowing agent or the use of supercritical CO₂ are other embodiments.

When the screw extruder 42 is used, the parameters of the extrusion include the rotational speed of the screw/s, feeding rates of the ingredients, the temperature distributions in the die and the barrel sections, the flow rates all of which can be altered as a function of time during the process to generate time dependent changes in the porosity and the composition distributions in the multi-layered extrudate 52 and the dimensions of the multi-layered extrudate 52. The number of layers can be increased by using additional extruders or delivery and pressurization apparati.

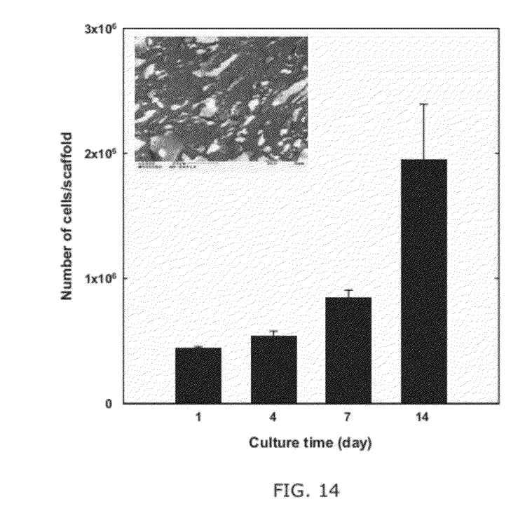

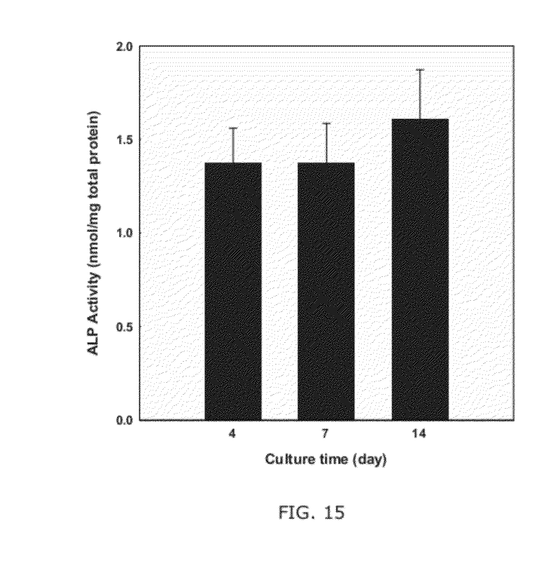

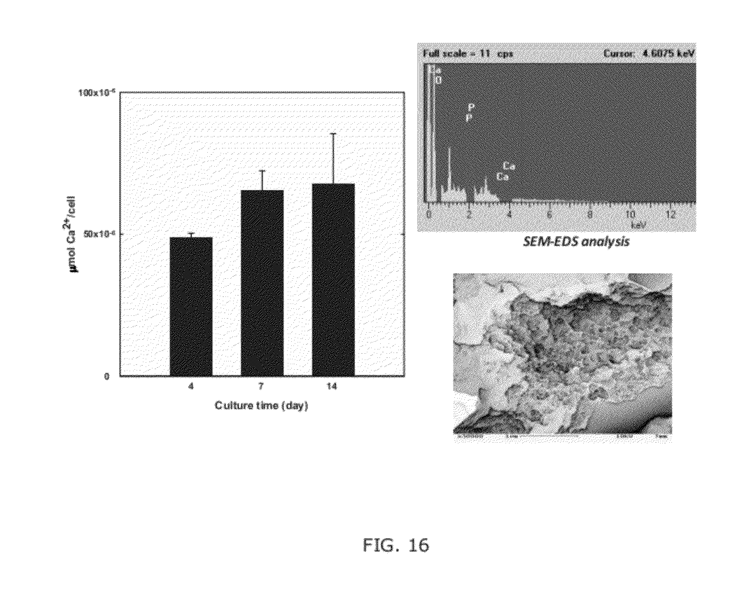

The implant 10 may be seeded with cells (stem cells to be differentiated or with chondrocytes/osteoblasts) for use as tissue engineering constructs, to give rise to tissue constructs that can then be implanted into the patient (e.g., at the fusion site). FIGS. 14-16 illustrate some of the cell proliferation results on prototype implants which are indicative of the osteoconductive and osteoinductive nature of the implants when they are employed as tissue engineering scaffolds. FIG. 14 illustrates cell attachment and proliferation on prototype implants of human fetal osteoblast (Hfob) cells seeded and cultured in vitro for 14 days, showing the biocompatibility of the implants. FIG. 15 illustrates Hfob cell differentiation on prototype implants. Alkaline phosphates activity (ALP) is indicative of osteoblastic phenotype development. FIG. 16 depicts mineralized matrix formation on prototype implants.

It will be understood that the embodiments described herein are merely exemplary and that a person skilled in the art may make many variations and modifications without departing from the spirit and scope of the invention. All such variations and modifications are intended to be included within the scope of the invention as defined in the appended claims.

What is claimed is:

1. A surgical implant, comprising:
a cage having a first porosity and a first modulus; and
a core bounded by said cage, said core having a second porosity that is higher than said first porosity of said cage, and said core having a second modulus that is lower than said first modulus of said cage.

2. The surgical implant of claim 1, wherein said implant is adapted to be implanted into a host bone, said first modulus of said cage is selected to substantially match a modulus of a cortical bone portion of the host bone, and said second modulus of said core is selected to substantially match a modulus of a cancellous bone portion of the host bone.

3. The surgical implant of claim 1, wherein said implant is adapted to be implanted into an intervertabral space.

4. The surgical implant of claim 1, wherein said implant is functionally graded in a transverse direction thereof.

5. The surgical implant of claim 1, wherein said implant is functionally graded in a longitudinal direction thereof.

6. The surgical implant of claim 5, wherein the implant includes a functional gradation of biphasic calcium phosphate in said longitudinal direction.

7. The surgical implant of claim 1, wherein said implant is functionally graded in a radial direction thereof.

8. The surgical implant of claim 1, wherein said cage encapsulates said core.

9. The surgical implant of claim 1, wherein said cage is concentrically formed around said core.

10. The surgical implant of claim 1, wherein said cage includes a first layer and a second layer, and wherein said core is sandwiched between said first and second layers of said cage.

11. The surgical implant of claim 1, wherein said cage and said core are made from a bioabsorbable polymer.

12. The surgical implant of claim 11, wherein said bioabsorbable polymer includes poly(caprolactone).

13. The surgical implant of claim 1, wherein said first porosity of said cage is about 74%, and said second porosity of said core is about 80%.

14. A method of making a surgical implant, comprising the steps of:
preparing a first formulation for a cage component of the implant within a first extruder;
preparing a second formulation for a core component of the implant within a second extruder;
extruding said first formulation through a co-extrusion die while simultaneously extruding said second formulation through said co-extrusion die so as to form an extrudate that includes said cage component and said core component bounded by said cage component;
conveying said extrudate from said co-extrusion die; and
forming said extrudate to a desired size and shape.

15. The method of claim 14, wherein the first formulation includes a bioabsorbable polymer and a first porogen, and the second formulation includes a bioabsorbable polymer and a second porogen.

16. The method of claim 15, wherein each of the first and second porogens includes poly(ethylene glycol) and sodium chloride.

17. The method of claim 16, wherein the first formulation comprises, by weight, about 20% poly(ethylene glycol), about 30% poly(caprolactone), and about 50% sodium chloride, and the second formulation comprises, by weight, about 36% poly(ethylene glycol), about 24% poly(caprolactone), and about 40% sodium chloride

18. An apparatus for making a surgical implant, comprising:
a first extruder for extruding a first formulation;
a second extruder for extruding a second formulation;
a co-extrusion die connected to said first extruder and said second extruder, said co-extrusion having a first channel in communication with said first extruder and a second channel in communication with said second extruder, said first and second channels converging with one another so as to form a transition zone, said first channel of said co-extrusion die is adapted to convey the first formulation through said transition zone, and said second channel of said co-extrusion die is adapted to convey the second formulation through said transition zone to form an implant extrudate.
